(19) United States
(12) Patent Application Publication
(10) Pub. No.: US 2013/0280235 A1
(43) Pub. Date: Oct. 24, 2013

(54) METHODS OF DIAGNOSIS AND TREATMENT FOR METABOLIC DISORDERS

(71) Applicant: DiaMedica Inc., Winnipeg (CA)

(72) Inventor: Mark S. Williams, Plymouth, MN (US)

(21) Appl. No.: 13/850,968

(22) Filed: Mar. 26, 2013

Related U.S. Application Data
(63) Continuation of application No. 12/374,872, filed on Sep. 29, 2009, now abandoned.
(60) Provisional application No. 60/909,829, filed on Apr. 3, 2007.
(60) Provisional application No. 60/820,379, filed on Jul. 26, 2006.

(51) Int. Cl. A61K 38/48; A61K 38/26

(52) U.S. Cl. CPC A61K 38/4853; A61K 38/26
USPC 424/94.64

(57) ABSTRACT

The invention relates to pharmaceutical compositions comprising tissue kallikrem (TK), and optionally a diabetes drug, a method of screening for a metabolic disorder by determining the concentration of TK and insulin in a biological sample from a test subject, a method of screening for a therapeutic agent for the treatment or prevention of a metabolic disorder, and a method for treating or preventing a metabolic disorder using a pharmaceutical composition comprising TK.

### CROSS-REFERENCE TO RELATED APPLICATIONS

This application claims priority from U.S. provisional application No. 60/820,379, filed Jul. 26, 2006 and U.S. provisional application No. 60/909,829, filed Apr. 3, 2007; the disclosures of which are hereby incorporated by reference.

### FIELD OF INVENTION

The present invention relates to methods of diagnosing metabolic disorders, and in particular insulin resistance and diabetes, and compounds for treating the same.

### BACKGROUND

Kallikrein is a group of proteases widely distributed in the plasma and tissues of animals, and is known to participate in an enzyme reaction system called the kallikrein-kinin system. The kallikrein-kinin system plays an important role in the regulation of functions in vivo.

There are two types of kallikrein, tissue or glandular kallikrein and plasma kallikrein. While both tissue kallikrein and plasma kallikrein are involved in kinin production, the two enzymes differ in many aspects including their genes of origin, molecular weight, amino acid sequences, substrates, and peptide products.

The tissue kallikrein-kinin system involves a series of enzyme reactions. Within the tissue kallikrein-kinin system, it is believed that tissue kallikrein is a serine protease which cleaves low-molecular-weight kininogen resulting in the release of kallidin (lysl-bradykinin). Kallidin can then be converted to bradykinin. Studies have also shown that tissue kallikrein may also cleave high-molecular-weight kininogen (herein after abbreviated as HMWK) (Moreau, M.E., Garbacki, N., Molinaro, G., Brown, N. J., and Marceau, F. (2005) The Kallikrein-Kinin System: Current and Future Pharmacological Targets. J Pharmacol Sci. 99:6-38).

The kallikrein-kinin system is in close relationship with various other enzymatic reaction systems such as the renin-angiotensin system, the blood clotting system, the fibrinolysis system, the complement system as well as the catecholamine and arachidonic acid cascades, which are mainly related to prostaglandins, leukotrienes and thromboxanes. Accordingly, the kallikrein-kinin system is closely associated with blood pressure regulating action and blood clotting-fibrinolysis-complement system action. Bioregulation and an improving action for peripheral circulation by various physiologically active substances produced by an arachidonic acid cascade are also related to the plasma kallikrein-kinin system.

Kinins, such as bradykinin, are produced in the kallikrein-kinin system. Kinins exhibit various physiological actions such as inducing a decrease in blood pressure due to dilation of peripheral blood vessels, promotion of permeability of blood vessels, contraction or relaxation of smooth muscle, induction of pain, induction of inflammation, migration of leucocytes, and liberation of catecholamine from the adrenal cortex.

Bradykinin is known to increase insulin sensitivity and has been suggested as a treatment for diabetes. For example, U.S. Pat. No. 4,146,613 teaches an orally administered anti-diabetes drug comprising a sulfyl urea and bradykinin. U.S. Pat. No. 4,150,121 teaches an injectable composition for treating diabetes comprising bradykinin and insulin.

The kallikrein-kinin system is also suggested to be under insulin control (Ottlecz, A., Koltai, M. and Gecse, A. (1979). Plasmakinin System in Alloxan Diabetic Rats. Current concepts in kinin research. Proceedings of the Satellite Symposium of the 7^{th} International Congress of Pharmacology. (pp 57-64) Oxford, England: Pergamon Press). These authors show that alloxan-induced type I diabetic rats, who are insulin deficient, have high kininogen levels in both plasma and tissue.

However, until the recent work of the present inventors, no one has contemplated the use of tissue kallikrein or kallikrein capable of cleaving low and/or high molecular weight kininogen for treating metabolic disorders such as insulin resistance and diabetes.

### SUMMARY OF INVENTION

In a first aspect, the present invention provides a method of screening a metabolic disorder comprising the steps of: (a) determining the concentration of a biomarker in a biological sample taken from a test subject, said biomarker selected from a group consisting of tissue kallikrein (KLKI), variants thereof, or biologically active fragments thereof, kininogen, or a combination thereof; and (b) comparing the concentration of the biomarker with a reference biomarker value range; wherein a determined biomarker concentration is outside the reference biomarker value range identifies a individual as affected with the metabolic disorder.

In an embodiment of the invention, the method of screening a metabolic disorder, further comprises the steps of: (c) determining the concentration of insulin in the biological sample taken from the test subject; and (d) comparing the concentration of insulin with a reference insulin value range; wherein, a determined biomarker concentration is greater than the reference biomarker value range and a determined insulin concentration is less than the reference insulin value range identifies a individual as affected with type 1 diabetes.

In another embodiment of the invention, the method of screening a metabolic disorder, further comprises the steps of: (c) determining the concentration of insulin in the biological sample taken from the test subject; and (d) comparing the concentration of insulin with a reference insulin value range; wherein, a determined biomarker concentration is less than the reference biomarker value range and a determined insulin concentration is greater than the reference insulin value range identifies a individual as affected with type 2 diabetes.

In a still further embodiment of the invention, the kininogen is high molecular weight kininogen.

In a further embodiment of the invention the kininogen is low molecular weight kininogen.

In yet a further embodiment of the invention, the test subject is human.

In another embodiment of the invention, the biological sample is blood.

In another embodiment of the invention, the biological sample is urine

In a further embodiment of the invention, the concentration of the biomarker is determined using a method selected from a group consisting of: immunoassay, liquid chromatography, gas chromatography, mass spectrometry, and a combination thereof.

In a still further embodiment of the invention, the concentration of insulin is determined using a method selected from a group consisting of: immunoassay, liquid chromatography, gas chromatography, mass spectrometry, and a combination thereof.

In a second aspect, the invention provides a pharmaceutical composition comprising tissues kallikrein (K1K1), a variant thereof, or a biologically active fragment thereof and a pharmaceutically acceptable carrier.

In a further embodiment of the invention, the tissue kallikrein (KLK1) is porcine tissue kallikrein.

In a further embodiment of the invention, the tissue kallikrein (KLK1) is human tissue kallikrein.

In an aspect, the invention provides a pharmaceutical composition comprising an ACE inhibitor, a cholinergic agonist and a pharmaceutically acceptable carrier.

In an embodiment of the invention, the ACE inhibitor is selected from a group consisting of: benazepril, captopril, cilazapril, enalapril, enalaprilat, fosinopril, lisinopril, moexipril, perindopril, quinapril, ramipril, trandolapril, and a mixture thereof.

In a further embodiment of the invention, the cholinergic agonist is selected from a group consisting of: acetylcholine, methacholine, bethanechol, BIBN 99, DIBD, SCH-57790, SCH-217443, SCH-72788, arecoline, an arecoline analogue, xanomeline, alvameline, milameline, RU 47213, sabcomeline, PD-151832, CDD-0034-C, CDD-0102, a spiropiperidine, a spiroquinuclidine, muscarine, cis-dioxolane, RS86, AF-30, ocvimelinc, AF150(S), AF267B, SDZ 210-086, YM-796, a rigid analogue of acetylcholine, acclidine, tasaclidine, oxotremorine, an oxotremorine analogue, pilocarpine, a pilocarpine analogue, thiopilocarpine, and a mixture thereof.

In a further aspect, the invention provides a pharmaceutical composition comprising: (a) tissue kallikrein (KLK1), a variant or a biologically active fragment thereof, (b) at least one diabetes drug, and (c) a pharmaceutically acceptable carrier.

In an embodiment of the invention, the at least one diabetes drug is selected from a group consisting of: an antioxidant, insulin, an insulin analogue, an α-adrenergic receptor antagonist, a β-adrenergic receptor antagonist, a non-selective adrenergic receptor antagonist, a sulphonylurea, a biguanide agent, a benzoic acid derivative, an α-glucosidase inhibitor, a thiazolidinedione, a phosphodiesterase inhibitor, a cholinesterase antagonist, a glutathione increasing compound, incretins or incretin mimetics.

In a further aspect, the incretin or incretin mimetic is selected from the group comprising: glucagon like peptide 1 (GLP-1), glucagon like peptide 2 (GLP-2), glucagon like peptide analogues or exenatide.

In a further aspect, the invention provides a method for the prevention or treatment of a metabolic disorder comprising administering a therapeutically effective amount of a pharmaceutical composition according to the invention to a subject in need thereof.

In a further aspect, the invention provides a method for the prevention or treatment of a metabolic disorder comprising administering a therapeutically effective amount of tissue kallikrein (KLK1) a variant thereof or a biologically active fragment thereof.

In an embodiment of the invention, the method for the prevention or treatment of a metabolic disorder comprises administering a therapeutically effective amount of KLK1.

In a further aspect, the invention provides use of tissue kallikrein (KLK1), a variant thereof or a biologically active fragment thereof, for treatment and prevention of a metabolic disorder, in a patient in need thereof.

In a further aspect, the invention provides use of tissue kallikrein (KLK1), a variant thereof, or a biologically active fragment thereof for the preparation of a medicament for treating and preventing a metabolic disorder, wherein said medicament comprises a therapeutically effective amount of tissue kallikrein (KLK1), a variant thereof, or a biologically active fragment thereof.

In an embodiment of the invention, KLK1 is used for the preparation of a medicament for treating and preventing a metabolic disorder.

In a further aspect, the invention provides use of an ACE inhibitor and a cholinergic agonist, for treatment and prevention of a metabolic disorder, in a patient in need thereof.

In an embodiment of the invention, the ACE inhibitor is selected from a group consisting of: benazepril, captopril, cilazapril, enalapril, enalaprilat, fosinopril, lisinopril, moexipril, perindopril, quinapril, ramipril, trandolapril, and a mixture thereof.

In another embodiment of the invention, the cholinergic agonist is selected from a group consisting of: acetylcholine, methacholine, bethanechol, BIBN 99, DIBD, SCH-57790, SCH-217443, SCH-72788, arecoline, an arecoline analogue, xanomeline, alvameline, milameline, RU 47213, sabcomeline, PD-151832, CDD-0034-C, CDD-0102, a spiropiperidine, a spiroquinuclidine, muscarine, cis-dioxolane, RS86, AF-30, ocvimeline, AF150(S), AF267B, SDZ 210-086, YM-796, a rigid analogue of acetylcholine, acclidine, tasaclidine, oxotremorine, an oxotremorine analogue, pilocarpine, a pilocarpine analogue, thiopilocarpine, and a mixture thereof.

In a further aspect, the invention provides a kit for use in treatment and prevention of a metabolic disorder, said kit comprising: (a) individual dosage forms of a pharmaceutical composition according to the invention; and (b) instructions for administration of the pharmaceutical composition to a subject in need thereof.

In a further aspect, the invention provides a kit for use in treatment and prevention of a metabolic disorder, said kit comprising: (a) individual dosage forms of tissue kallikrein (KLK1) and (b) instructions for administration of the dosage form to a subject in need thereof.

In a further aspect, the invention provides a method of screening for a therapeutic agent for treatment or prevention of a metabolic disorder resulting from aberrant expression of a polynucleotide sequence encoding KLK1 or a variant or biologically active fragment thereof, the method comprising the steps of: (a) contacting a reporter construct under the control of a kallikrein promoter with a test molecule or compound, or a library of test molecules or compounds, under conditions to allow specific binding and/or interaction; and (b) detecting the level of expression of the reporter construct; wherein an alteration in the level of expression to a control indicates a potential therapeutic activity.

In an aspect, the invention provides a method of screening for a therapeutic agent for treatment or prevention of a metabolic disorder resulting from altered biological activity of a tissue kallikrein (KLK1), a variant thereof or biologically active fragment thereof, the method comprising the steps of: (a) contacting a tissue kallikrein (KLK1), variant thereof or biologically active fragment thereof with a test molecule or compound, or a library of test molecules or compounds, under conditions to allow specific binding and/or interaction; and (b) detecting the level of specific binding and/or interaction, wherein an alteration in the level of interaction relative to a control indicates a potential therapeutic activity.

In an embodiment of the invention, the metabolic disorder is selected from a group consisting of: insulin resistance, pre-diabetes, diabetes, impaired glucose tolerance, impaired glucose metabolism, hyperglycemia, hyperinsulinaemia, and syndrome X.

In another embodiment of the invention, the library of test molecules or compounds is selected from the group consisting of DNA molecules, peptides, agonists, antagonists, monoclonal antibodies, immunoglobulins, small molecule drugs and pharmaceutical agents.

### BRIEF DESCRIPTION OF THE FIGURES

FIG. 1 illustrates the chemical structure for various M2 muscarinic antagonists.

FIG. 2 illustrates the chemical structure for various areoline-analogue muscarinic agonists.

FIG. 3 illustrates the chemical structure for various spiropiperidines and spiroquinuclidines having muscarinic activity.

FIG. 4 illustrates the chemical structure for various rigid analogues of acetylcholine having muscarinic activity.

FIG. 5 illustrates the chemical structure for various oxotremorine and pilocarpine muscarinic agonists.

### DETAILED DESCRIPTION

The present inventors have now determined that the kallikrein-kinin system plays an apparent separate role in the regulation of insulin responsiveness. The present inventors have determined that alterations to the kallikrein-kinin system and in particular alterations to the tissue kallikrein-kinin system results in impaired insulin sensitivity which is not further modulated by hepatic muscarinic cholinergic blockade. The present inventors have further determined that insulin insensitivity is correlated with alterations to tissue kallikrein expression and/or biological activity. Thus, while the present invention is not restricted to any particular model or mechanism of action, it is believed that the kallikrein-kinin system, and in particular, the tissue kallikrein-kinin system, modulates insulin sensitivity.

“Tissue kallikrein” or “KLK1” is a serine protease that is primarily noted for its role in controlling hypertension through its cleavage of kininogen into lysyl-bradykinin (Yousef et al., Endocrine Rev. 2001; 22: 184-204). As there are a large number of enzymes in the KLK family, the inventors believe that KLK1 appears to be a ubiquitous or multiple target acting enzyme, in addition to its recognized role in hypertension regulation, and as such may specifically play an important role in insulin sensitivity and glucose control. As used herein, the term “tissue kallikrein” or “KLK1” is synonymous with the following terms: callicrein, glumorin, padreatin, padutin, kallidinogenase, bradykininogenase, pancreatic kallikrein, onokrein P, diliminal D, depot-Padutin, urokallikrein or urinary kallikrein.

Tissue kallikrein has the following sequences: (SEQ ID No. 1)

NP_001001911 GI: 50054435 Sus scrofa
1-17 signal peptide
18-24 propeptide
25-263 mature peptide
>gi|50054435|ref|NP_001001911.1|kallikrein 1
[Sus scrofa]
MWSLVMRLALSLAGTGAAPPIQSRIIGGRECEKDSHPWQVAIYHYSSFQC
GGVLVDPKWVLTAAHCKNDNYQVWLGRHNLFENEVTAQFFGVTADFPHPG
FNLSLLKNHTKADGKDYSHDLMLLRLQSPAKITDAVKVLELPTQEPELGS
TCQASGWGSIEPGPDDFEFPDEIQCVELTLLQNTFCADAHPDKVTESMLC
AGYLPGGKDTCMGDSGGPLICNGMWQGITSWGHTPCGSANKPSIYTKLIF
YLDWINDTITENP

### Or (SEQ ID No. 2)

NP_002248 GI: 4504875 Homo sapiens
1-18 signal peptide
19-24 propeptide
25-262 mature peptide
>gi|4504875|ref|NP_002248.1|kallikrein 1
preproprotein [Homo sapiens]
MWFLVLCLALSLGGTGAAPPIQSRIVGGWECEQHSQPWQAALYHFSTFQC
GGILVHRQWVLTAAHCISDNYQLWLGRHNLFDDENTAQFVHVSESFPHPG
FNMSLLENHTRQADEDYSHDLMLLRLTEPADT1TDAVKVVELPTEEPEVG
STCLASGWGSIEPENFSFPDDLQCVDLKILPNDECKKAHVQKVTDFMLCV
GHLEGGKDTCVGDSGGPLMCDGVLQGVTSWGYVPCGTPNKPSVAVRVLSY
VKWIEDTIAENS

### Diagnostics

The present inventors have determined that alterations to the kallikrein-kinin system, and in particular the tissue kallikrein-kinin system is accountable for decreased insulin sensitivity observed in metabolic disorders such as type 1 and type 2 diabetes. In particular, the present inventors have determined that alterations to tissue kallikrein expression and/or biological activity are strongly correlated with decreased insulin sensitivity. Accordingly, the kallikrein, and its substrate kininogen, can be used as biomarkers for screening metabolic disorders characterized by decreased insulin sensitivity.

“Biomarkers” as used herein refers to a molecule whose absence or presence indicates an alteration in physiology from normal.

“Metabolic disorders” as used herein, refers to any metabolic disorder directly or indirectly resulting from impaired insulin sensitivity and/or glucose utilization. Examples of metabolic disorders include but are not limited to insulin resistance, pre-diabetes, diabetes, impaired glucose intolerance, impaired glucose metabolism, hyperglycemia, hyperinsulinaemia, and syndrome X.

“Screening” as used herein refers to a procedure used to evaluate a subject for the presence of a disorder characterized by expression of one or more biomarkers, as described above. It is not required that the screening procedure be free of false positives or false negatives, as long as the screening procedure is useful and beneficial in determining which of those individuals within a group or population of individuals are affected with a particular disorder. The screening methods disclosed herein may be diagnostic and/or prognostic methods and/or may be used to monitor patient therapy.

A “diagnostic method”, as used herein, refers to a screening procedure that is carried out to identify those subjects that are affected with a particular disorder.

A “prognostic method” refers to a method used to help predict, at least in part, the course of a disease. Alternatively stated, a prognostic method may be used to assess the severity of the disease. For example, the screening procedure disclosed herein may be carried out to both identify an affected individual, to evaluate the severity of the disease, and/or to predict the future course of the disease. Such methods may be useful in evaluating the necessity for therapeutic treatment, what type of treatment to implement, and the like. In addition, a prognostic method may be carried out on a subject previously diagnosed with a particular disorder when it is desired to gain greater insight into how the disease will progress for that particular subject (e.g., the likelihood that a particular patient will respond favorably to a particular drug treatment, or when it is desired to classify or separate patients into distinct and different sub-populations for the purpose of conducting a clinical trial thereon).

The terms “quantifying the concentration” or “determining the concentration,” as used herein, refer to measurement of the concentration or level of the analyte in the indicated sample. Typically, an absolute or relative numerical value will be assigned to the concentration of the analyte in the sample as a result of the quantifying or determining step. Any suitable method known in the art may be used to quantify or determine the concentration of one or more biomarkers in a biological sample according to the present invention, as described in more detail hereinbelow.

Methods of “quantifying” or “determining” the concentration of a biomarker encompass both quantitative and or semi-quantitative methodologies, also as described in more detail below.

A “quantitative” method is one that assigns an absolute or relative numerical value to the concentration of the analyte in the biological sample.

A “semi-quantitative” method is one that indicates that the concentration of the analyte is above a threshold level, but does not assign an absolute or relative numerical value.

In general, the methods disclosed herein have both veterinary and medical applications. Accordingly, subjects may be humans, simians, canines, felines, equines, bovines, ovines, caprines, porcines, lagomorphs, rodents, avians, and the like. Typically, however, subjects according to the present invention will be human subjects.

As used herein, the “biological sample” may comprise any suitable body fluid, cells, or tissue (including cultured cells and tissue) in which one or more of the biomarkers may be detected in. Preferably, the biological sample will be blood or urine.

The invention provides a method of screening for metabolic disorders by detecting biomarkers which correlate with altered insulin sensitivity. The inventors have determined that levels of tissue kallikrein and kininogen are strongly correlated with altered insulin sensitivity. Accordingly, the present invention provides a method for screening metabolic disorders including but not limited to insulin resistance and diabetes which involves the detection of these biomarkers.

The screening method comprises the steps of: (a) determining the concentration of a biomarker in a biological sample taken from a test subject, said biomarker selected from a group consisting of: tissue kallikrein or a variant or and biologically active fragment thereof, kininogen, or a combination thereof; and (b) comparing the concentration of the biomarker with a reference biomarker value range; wherein a determined biomarker concentration is outside the reference biomarker value range identifies a individual as affected with the metabolic disorder.

The method encompasses the use of tissue kallikrein and both low molecular weight kininogen and high molecular weight kininogen. In one preferred embodiment, the biomarker is preferably tissue kallikrein. In another preferred embodiment, the biomarker is preferably high molecular weight kininogen.

Reference values can be determined by measuring biomarker levels in subjects with normal and impaired insulin sensitivity using statistical methods known in the art.

The inventors have determined that type 1 diabetes is characterized by higher levels of kallikrein-kinin activity and decreased levels of insulin whereas type 2 diabetes is characterized by lower levels of kallikrein-kinin activity and increased levels of insulin. Thus, the invention further provides a method for differentially screening type 1 versus type 2 diabetes. In this embodiment of the invention, the preferred biomarker is tissue kallikrein. and the method of screening further comprises the step of determining the concentration of insulin in the biological sample taken from the test subject; and comparing the concentration of insulin with a reference insulin value range.

The present invention further finds use in methods of monitoring the clinical course of a test subject that has already been positively diagnosed as affected with a disorder characterized by the aberrant levels of any of the biomarkers. The level of any of the biomarkers may correlate with the clinical state of the affected subject, i.e. decreased expression of tissue kallikrein may correlate with increase severity of insulin resistance. Thus, the levels of the biomarkers could be used as an index of treatment efficacy and the clinical condition of the patient.

Accordingly, the present invention further encompasses methods of monitoring the clinical status of a subject with a disorder characterized by the levels of one or more biomarkers. The clinical condition of the subject may be monitored to determine the efficacy of a treatment regime, e.g., drug or dietary therapy. For example, if levels of the biomarker suggest that the current therapeutic regime is not effective, it may be determined to initiate an altered course of treatment. Alternatively, the condition of the subject may be monitored to determine whether to commence or re-initiate treatment of the subject.

The inventive screening methods disclosed herein may be carried out using any suitable methodology that detects the presence or absence of the biomarkers, determines the concentration of the biomarkers in a biological sample (as described above). Illustrative methods include, but are not limited to, chromatographic methods (e.g., high performance liquid chromatography), immunoassay (e.g., immunoaffinity chromatography, immunoprecipitation, radioimmunoassay, immunofluorescence assay, immunocytochemical assay, immunoblotting, enzyme-linked immunosorbent assay (ELISA) and the like), liquid chromatography-mass spectrometry; gas chromatography-mass spectrometry, time-of-flight mass spectrometry, tandem mass spectrometry, and combinations of these mass spectrometry techniques with immunopurification.

Preferred methods will be simple, rapid, accurate, sensitive, and preferably minimize interfering signals from molecules other than the biomarkers. When used as a method for mass screening, it is further preferred that the methodology is compatible with existing screening assays and is adaptable to automation and high through-put screening of samples.

The methods may be completely manual, alternatively and preferably, they are partially or completely automated. Screening programs to evaluate a large number of samples (e.g., community screening programs) will generally be at least partially automated to facilitate high throughput of samples. Typically, for example, the data will be captured and analyzed using an automated system. In other preferred high throughput methods, arrays or micro-arrays of spotted biological samples (e.g., blood, urine) may be analyzed concurrently.

In one embodiment of the invention, MS/MS is a preferred methodology for carrying out the inventive methods described above. The concept of MS/MS for analysis of mixtures using triple quadrupole mass spectrometers was originated by Yost and Enke, Tandem quadrupole mass spectrometry. In: Tandem Mass Spectrometry, F. W. McLafferty (Ed.), Wiley & Sons, New York, (1983), pp. 175-195. For the selective detection of compounds of a similar structural type, either a precursor ion scan function to identify the molecular species that fragment to a common product ion, or a constant neutral loss scan function to identify ions that lose a common fragment, or a multiple reaction monitoring where selected precursor and product ions only are detected is employed. Addition of appropriate internal standards, such as stable isotope-labeled analogs, to the biological matrix before work-up and analysis facilitates accurate quantification of the target analytes.

Any suitable MS/MS methodology known in the art may be employed, including, but not limited to triple quadrupole mass spectrometry and hybrid mass spectrometry methods that combine quadrupole and time-of-flight mass spectrometers. Ion traps and ion cyclotron resonance mass spectrometers can also be employed.

Alternatively, immunoassays can also be used to detect the absence or the presence of one or more biomarkers using antibodies. The term “antibodies” as used herein refers to all types of immunoglobulins, including IgG, IgM, IgA, IgD, and IgE. The antibodies may be monoclonal or polyclonal and may be of any species of origin, including (for example) mouse, rat, rabbit, horse, or human, or may be chimeric antibodies. See, e.g., M. Walker et al., Molec. Immunol. 26, 403-11 (1989). The antibodies may be recombinant monoclonal antibodies produced according to the methods disclosed in Reading U.S. Pat. No. 4,474,893, or Cabilly et al., U.S. Pat. No. 4,816,567. The antibodies may also be chemically constructed by specific antibodies made according to the method disclosed in Segal et al., U.S. Pat. No. 4,676,980.

In order to quantify the concentration or a particular biomarker, a monoclonal antibody specific for the biomarker can be attached to a solid surface such as a plate, tube, bead, or particle. Preferably, the antibody is attached to the well surface of a multi-well ELISA plate. 100 μl sample of blood is added to the solid phase antibody. The sample is incubated for 2 hrs at room temperature. Next the sample fluid is decanted, and the solid phase is washed with buffer to remove unbound material. 100 μl of a second monoclonal antibody (to a different determinant on the subject polypeptide/protein) is added to the solid phase. This antibody is labeled with a detector molecule (e.g., ¹²⁵I, enzyme, fluorophore, or a chromophore) and the solid phase with the second antibody is incubated for two hrs at room temperature. The second antibody is decanted and the solid phase is washed with buffer to remove unbound material. The amount of bound label, which is proportional to the amount of biomarker present in the sample, is quantitated. Other types of immunoassays known in the art can also be employed.

Where it is desirable to measure insulin concentration, any suitable method described above can be employed. Preferably, plasma insulin can be assayed using a radioimmunoassay, such method being well known in the art.

### Drug Screening

The present invention further provides methods for screening therapeutic agents for use in the treatment and prevention of metabolic disorders characterized by insulin insensitivity. The methods of screening are based on the inventors' discovery that alterations to the kallikrein-kinin system and in particular alterations to tissue kallikrein expression and/or biological activity are strongly correlated with decreased insulin sensitivity.

The phrase “library of test molecules or compounds” is used herein to include libraries containing DNA molecules, peptides, agonists, antagonists, monoclonal antibodies, immunoglobulins and/or pharmaceutical agents. These may include new or already known molecules or compounds. Furthermore, the terms “monoclonal antibodies” and “immunoglobulins” used herein include fragments or derivatives thereof.

The term “reporter construct”, herein, encompasses a target gene linked in frame to another sequence to provide a coding unit whose product is easily assayed. Examples of reporter genes include, but are not limited to, β-galactosidase, luciferase, green fluorescent protein (GFP), enhanced green fluorescent protein (EGFP), Ds Red fluorescent protein, far-red fluorescent protein (Hc-red), secreted alkaline phosphatase (SEAP), chloramphenicol acetyltransferase (CAT), neomycin etc.

The test compounds according to the present invention can be obtained using any of the numerous approaches in combinatorial library methods known in the art, including: biological libraries, aptially addressable parallel solid phase or solution phase libraries, synthetic library methods requiring deconvolution, the “one-bead-one-compound” library method, and synthetic library methods using affinity chromatography selection. The biological library approach is limited to peptide libraries, while the other four approaches are applicable to peptide, non peptide oligomer or small molecule libraries of compounds (Bindseil et al. (2001) Drug Discov. Today 6, 840-847; Grabley et al. (2000) Ernst Schering Res. Found. Workshop. pp. 217 252; Houghten et al. (2000) Drug Discov. Today 5, 276 285; Rader, C. (2001) Drug Discov. Today 6, 36 43).

Examples of methods for the synthesis of molecular libraries can be found in the art, for example in: DeWitt et al. (1993) Proc. Natl. Acad. Sci. USA 90, 6909-6913; Erb et al. (1994) Proc. Natl. Acad. Sci. USA 91, 11422-11426; Gallop et al. (1994) J. Med. Chem. 37, 1233-1251; Gordon et al. (1994) J. Med. Chem. 37, 1385 1401.

Libraries of compounds may be presented in solution (e.g., Houghten (1992) Biotechniques 13, 412-421), or on beads (Lam et al. (1991) Nature 354, 82-84), chips (Fodor et al. (1993) Nature 364, 555-556), bacteria (U.S. Pat. No. 5,223,409, published June 1993), spores [U.S. Pat. Nos. 5,571,698 (published in November 1996); 5,403,484 (published in April 1995); and 5,223,409 (published in June 1993)], plasmids (Cull et al. (1992) Proc. Natl. Acad. Sci. USA 89, 1865-1869) or phages (Scott and Smith (1990) Science. 249, 386-390; Devlin et al. (1990) Science. 249, 404-406; Cwirla et al. (1990) Proc. Natl. Acad. Sci. USA 87, 6378 6382; Felici et al. (1991) J. Mol. Biol. 222, 301-310).

In one aspect, the invention provides a method for screening for a therapeutic agent for treatment or prevention of a metabolic disorder resulting from aberrant expression of a polynucleotide sequence encoding tissue kallikrein, the method comprising the steps of: (a) contacting a reporter construct under the control of a tissue kallikrein promoter with a test molecule or compound, or a library of test molecules or compounds, under conditions to allow specific binding and/or interaction; and (b) detecting the level of expression of the reporter construct, wherein an alteration in the level of expression to a control indicates a potential therapeutic activity.

A tissue kallikrein promoter can be isolated by screening a genomic library with a kallikrein cDNA; preferably containing the 5′ end of the cDNA. In a preferred embodiment, the kallikrein promoter is a tissue kallikrein promoter. The gene for tissue kallikrein has been identified in a number of species including human (see NCBI Accession No. AAB34120).

A portion of said kallikrein promoter, typically from 20 to about 500 base pairs long is then cloned upstream of a reporter gene, e.g., a β-galactosidase, luciferase, green fluorescent protein (GFP), enhanced green fluorescent protein (EGFP), Ds-Red fluorescent protein, far-red fluorescent protein (Hc-red), secreted alkaline phosphatase (SEAP), chloramphenicol acetyltransferase (CAT), neomycin gene, in a plasmid. This reporter construct is then transfected into cells, e.g., mammalian cells. The transfected cells are distributed into wells of a multi-well plate and various concentrations of test molecules or compounds are added to the wells. After several hours of incubation, the level of expression of the reporter construct is determined according to methods known in the art. A difference in the level of expression of the reporter construct in transfected cells incubated with the test molecule or compound relative to transfected cells incubated without the test molecule or compound will indicate that the test molecule or compound is capable of modulating the expression of a kallikrein.

In another aspect, the invention provides a method of screening for a therapeutic agent for treatment or prevention of a metabolic disorder resulting from altered biologically activity of tissue kallikrein, the method comprising the steps of: (a) contacting tissue kallikrein or an active fragment thereof with a test molecule or compound, or a library of test molecules under conditions to allow specific binding and/or interaction to provide a bound complex; and (b) adding tissue kallikrein substrate to the bound complex and measuring kallikrein activity, wherein an alteration in the level of kallikrein activity relative to a control indicates a potential therapeutic activity.

In a preferred embodiment the substrate is kininogen. The type of kininogen used will depend on the type of kallikrein used. Other kallikrein substrates known in the art, including synthetic substrates, can also be used to practice the method.

The method may be practiced using biological samples such as blood or plasma or urine samples, which contain endogenous tissue kallikrein. Alternatively, the method may be practiced using hosts cells which have been transformed to express a functional kallikrein. Where the kallikrein employed is tissue kallikrein, it is preferable to express a biologically active fragment of the tissue kallikrein.

In another embodiment of the invention, a cell free system comprising purified tissue kallikrein or biologically active fragments thereof, can be used to practice the screening method. It may be desirable to immobilize the kallikrein or kallikrein fragment on a solid matrix.

### Pharmaceutical Compositions

### Compositions Comprising Tissue Kallikrein or an Active Fragment

The term “biologically active fragment” refers to smaller protions of the KLK1 polypeptide that retains the activity of the full length KLK1 polypeptide.

A “variant” or “mutant” of a starting or reference polypeptide is a polypeptide that 1) has an amino acid sequence different from that of the starting or reference polypeptide and 2) was derived from the starting or reference polypeptide through either natural or artificial (manmade) mutagenesis. Such variants include, for example, deletions from, and/or insertions into and/or substitutions of, residues within the amino acid sequence of the polypeptide of interest. A variant amino acid, in this context, refers to an amino acid different from the amino acid at the corresponding position in a starting or reference polypeptide sequence (such as that of a source antibody or antigen binding fragment). Any combination of deletion, insertion, and substitution may be made to arrive at the final variant or mutant construct, provided that the final construct possesses the desired functional characteristics. The amino acid changes also may alter post-translational processes of the polypeptide, such as changing the number or position of glycosylation sites.

In another aspect of the invention, provided are pharmaceutical compositions which modulate the kallikrein-kinin system and in particular supplement endogenous levels of tissue kallikrein. The pharmaceutical compositions are particularly useful for the treatment and prevention of metabolic disorders characterized by insulin sensitivity and more preferably for the treatment and prevention of metabolic disorders characterized by impaired tissue kallikrein expression and/or biological activity.

The pharmaceutical composition comprises a tissue kallikrein or a biologically active fragment thereof and a pharmaceutically acceptable carrier.

In a preferred embodiment of the invention, the pharmaceutical composition comprises the native active form of tissue kallikrein or a fragment thereof which substantially retains the protease activity of native active tissue kallikrein. Such biologically active fragments include polypeptides comprising amino acids of the native protein.

### Compositions Comprising an ACE Inhibitor and a Cholinergic Agonist

The scientific literature and work herein demonstrates that bradykinin can increase insulin sensitivity. It has been shown that ACE inhibitors prevent the degradation of bradykinin (and related species) and subsequently also increase insulin sensitivity. The present inventors have shown that the use of a cholinergic agonist leads to the activation of the kallikrein-kinin system. Together, ACE inhibitors and cholinergic agonists substantially increase insulin sensitivity.

The present invention provides a further pharmaceutical composition useful for the treatment and prevention of metabolic disorders characterized by insulin insensitivity. The pharmaceutical composition comprises an ACE inhibitor, a cholinergic agonist and a pharmaceutically acceptable carrier. The pharmaceutical composition is based on the inventors' discovery that combined modulation of the kallikrein-kinin and hepatic parasympathetic systems results in synergistic improvements in insulin responsiveness as compared to the administration of either an ACE inhibitor or a cholinergic agonist alone.

Any suitable ACE inhibitor may be employed to practice the invention. Examples of suitable ACE inhibitors include but are not limited to: benazepril; captopril; cilazapril; enalapril; enalaprilat; fosinopril; lisinopril; moexipril; perindopril; quinapril; ramipril; trandolapril; or a mixture thereof. In a preferred embodiment, the ACE inhibitor is enalapril or lisinopril.

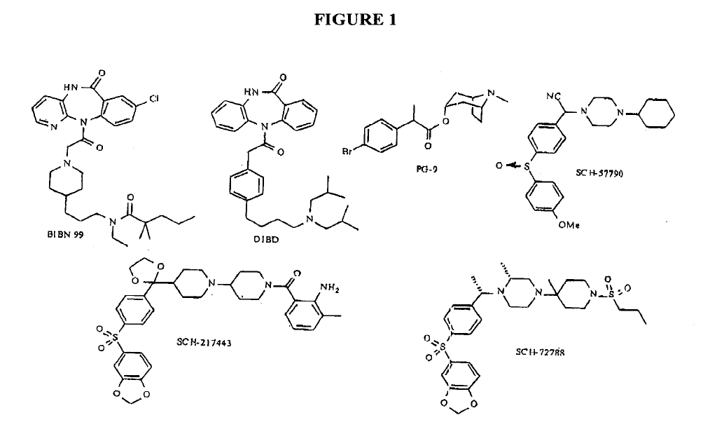

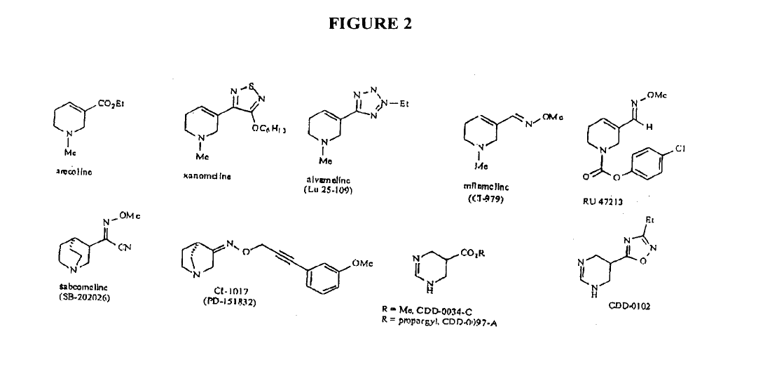

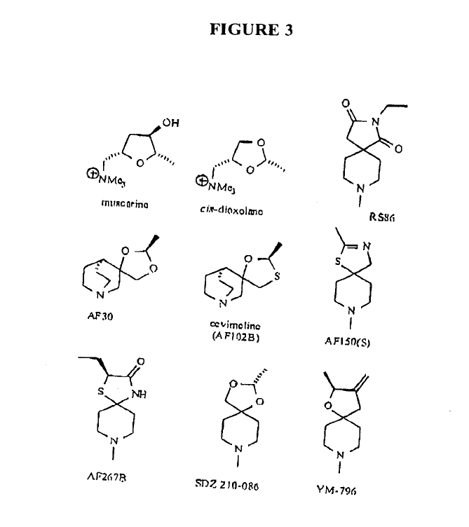

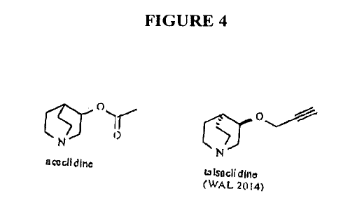

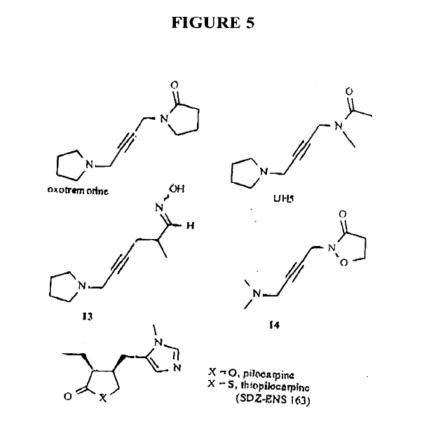

Any suitable cholinergic agonist may be employed to practice the invention. Examples of suitable cholinergic agonists, include, but are not limited to: acetylcholine, methacholine, bethanechol, BIBN 99 (FIG. 1), DIBD (FIG. 1), SCH-57790 (FIG. 1), SCH-217443 (FIG. 1), SCH-72788 (FIG. 1), arecoline (FIG. 2), an arecoline analogue (FIG. 2), xanomeline (FIG. 2), alvameline (FIG. 2), milameline (FIG. 2), RU 47213 (FIG. 2), sabcomeline (FIG. 2), PD-151832 (FIG. 2), CDD-0034-C (FIG. 2), CDD-0102 (FIG. 2), a spiropiperidine (FIG. 3), a spiroquinuclidine (FIG. 3), muscarine (FIG. 3), cis-dioxolane (FIG. 3), RS86 (FIG. 3), AF-30 (FIG. 3), ocvimeline (FIG. 3), AF150(S) (FIG. 3), AF267B (FIG. 3), SDZ 210-086 (FIG. 3), YM-796 (FIG. 3), a rigid analogue of acetylcholine (FIG. 4), acclidine (FIG. 4), tasaclidine (FIG. 4), oxotremorine (FIG. 5), an oxotremorine analogue (FIG. 5), pilocarpine (FIG. 5), a pilocarpine analogue (FIG. 5), or thiopilocarpine (FIG. 5). A nitrosylated form of any these compounds can also be employed. In an embodiment of the invention, the cholinergic agonist is preferably acetylcholine and more preferably bethanechol.

### Compositions Comprising Tissue Kallikrein and a Known Diabetes Drug

The present invention provides a further pharmaceutical composition useful for the treatment and prevention of metabolic disorders characterized by insulin insensitivity. The present invention provides novel pharmaceutical compositions comprising: a) tissue kallikrein, b) at least one diabetes drug and c) a pharmaceutically acceptable carrier.

As used herein, the term “diabetes drug” refers to any composition known in the art to be useful in the treatment or prevention of insulin resistance and diabetes. Examples of diabetes drugs which may be used to practice the invention, include but are not limited to:
(a) an antioxidant such as vitamin E, vitamin C, an isoflavone, zinc, selenium, ebselen, a carotenoid;
(b) an insulin or insulin analogue such as regular insulin, lente insulin, semilente insulin, ultralente insulin, NPH or humalog;
(c) an α-adrenergic receptor antagonist such as prazosin, doxazocin, phenoxybenzamine, terazosin, phentolamine, rauwolscine, yohimbine, tolazoline, tamsulosin, or terazosin;
(d) a β-adrenergic receptor antagonist such as acebutolol, atenolol, betaxolol, bisoprolol, carteolol, esmolol, metoprolol, nadolol, penbutolol, pindolol, propanolol, timolol, dobutamine hydrochloride, alprenolol, bunolol, bupranolol, carazolol, epanolol, moloprolol, oxprenolol, pamatolol, talinolol, tiprenolol, tolamolol, or toliprolol;
(e) a non-selective adrenergic receptor antagonist such as carvedilol or labetolol;
(f) a first generation sulphonylurea such as tolazamide, tolubtuamide, chlorpropamide, acetohexamide;
(g) a second generation sulphonylurea such as glyburide, glipizide, and glimepiride;
(h) a biguanide agent such as is metformin;
(i) a benzoic acid derivative such as replaglinide;
(j) a α-glucosidase inhibitor such as acarbose and miglitol;
(k) a thiazolidinedione such as rosiglitazone, pioglitazone, or troglitazone;
(l) a phosphodiesterase inhibitor such as anagrelide, tadalfil, dipyridamole, dyphylline, vardenafil, cilostazol, milrinone, theophylline, or caffeine;
(m) a cholineresterase antagonist such as donepezil, tacrine, edrophonium, demecarium, pyridostigmine, zanapezil, phospholine, metrifonate, neostigmine, or galathamine;
(n) a glutathione increasing compound such as N-acetylcysteine, a cysteine ester, L-2-oxothiazolidine-4-carboxolate (OTC), gamma glutamylcysteine and its ethyl ester, glytathtione ethyl ester, glutathione isopropyl ester, lipoic acid, cysteine, methionine, or S-adenosylmethionine; and
(o) incretin or incretin mimetics like GLP-1, GLP-2, glucagon like peptide analogues, such as DAC:GLP-1 (CJC-1131), Liraglutide, ZP10, BIM51077, LY315902, LY307161 (SR), and exenatide.

The pharmaceutical composition can be prepared with tissue kallikrein. The tissue kallikrein may be porcine tissue kallikrein or human tissue kallikrein. Preferably, the pharmaceutical composition is prepared using a kallikrein which cleaves high or low molecular weight kininogen.

### Pharmaceutical Formulations and Methods of Preparation

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

Suitable routes of administration may, for example, include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/orpolyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The pushfit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example, subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a co-solvent system comprising benzyl alcohol, a nonpolar surfactant, a water-miscible organic polymer, and an aqueous phase. Naturally, the proportions of a co-solvent system may be varied considerably without destroying its solubility and toxicity characteristics. Furthermore, the identity of the co-solvent components may be varied.

Alternatively, other delivery systems for hydrophobic pharmaceutical compounds may be employed.

Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients.

Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

### Methods of Treatment

In a further aspect, the invention provides a method of treating and preventing a metabolic disorder comprising the administration of a therapeutically effective amount of any of the pharmaceutical compositions according to the invention described above to a subject in need thereof.

In another aspect, the invention provides a method of treating and preventing a metabolic disorder comprising the administration of a therapeutically effective amount of tissue kallikrein or a variant or a biologically active fragment thereof to a subject in need thereof. In a preferred embodiment of the invention, the kallikrein is a tissue kallikrein in its active form.

In another aspect, the invention provides a method of treating and preventing a metabolic disorder comprising the administration of a therapeutically effective amount of tissue kallikrein and with at least one diabetes drug.

In another aspect of the invention the diabetes drug is GLP-1.

Examples of metabolic disorders which may be treated and prevented using pharmaceutical compositions according to the invention include but are not limited to: insulin resistance, pre-diabetes, diabetes, impaired glucose tolerance, impaired glucose metabolism, hyperglycemia, hyperinsulinaemia, and syndrome X.

By an “effective amount” or a “therapeutically effective amount” of a pharmacologically active agent is meant a nontoxic but sufficient amount of the drug or agent to provide the desired effect. In a combination therapy of the present invention, for example the co-administration of tissue kallikrein with an ACE inhibitor with a cholinergic agonist, an “effective amount” of one component of the combination is the amount of that compound that is effective to provide the desired effect when used in combination with the other components of the combination. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The therapeutic effective amount of any of the active agents encompassed by the invention will depend on number of factors which will be apparent to those skilled in the art and in light of the disclosure herein. In particular these factors include: the identity of the compounds to be administered, the formulation, the route of administration employed, the patient's gender, age, and weight, and the severity of the condition being treated and the presence of concurrent illness affecting the gastro-intestinal tract, the hepatobillary system and the renal system. Methods for determining dosage and toxicity are well known in the art with studies generally beginning in animals and then in humans if no significant animal toxicity is observed. The appropriateness of the dosage can be assessed by monitoring insulin resistance using the RIST protocol as set out in Lautt et al, 1998 or the standard euglycemic clamp procedure. Where the dose provided does not cause insulin resistance to decline to normal or tolerable levels, following at least three days of treatment the dose can be increased. The patient should be monitored for signs of adverse drug reactions and toxicity, especially with regard to liver function.

Where the method of treatment and prevention comprises the administration of tissue kallikrein, the preferred unit dosage is between 0.1 and 100 units per day and more preferably between 1 and 10 units per day.

Where the method of treatment and prevention comprises the administration of an ACE inhibitor and a cholinergic agonist, each component may be administered concurrently as a single formulation or serially as separate formulations.

The therapeutic effective unit dose of the ACE inhibitor will vary depending on the particular ACE inhibitor employed. Suitable dosage ranges for ACE inhibitors are known in the art. Where the ACE inhibitor employed is lisinopril, the preferred unit dosage is between 1 and 100 mg/day and more preferably, 20 mg/day. Where the ACE inhibitor employed is captopril, the preferred unit dosage is between 1 and 150 mg/day. Where the ACE inhibitor employed is enalapril, the preferred unit dosage is between 1 and 100 mg/day. Where the ACE inhibitor is ramipril, the preferred unit dosage is between 1.25 and 100 mg/day. Where the ACE inhibitor is trandolapril, the preferred unit dosage is between 1 and 4 mg/day.

The therapeutic effective dose of the cholinergic agonist also will vary depending on the particular cholinergic agonist used. Where the cholinergic agonist is acetylcholine or bethanechol, the dosage will be between 0.001 mg/kg and 100 mg/kg and preferably between 0.001 mg/kg and 1 mg/kg.

Although the present invention has been described with reference to illustrative embodiments, it is to be understood that the invention is not limited to these precise embodiments, and that various changes and modifications may be effected therein by one skilled in the art. All such changes and modifications are intended to be encompassed in the appended claims.

### EXAMPLES

### Example One

### Kallikrein Therapy Restores Insulin Responsiveness in Diabetic Animal Model

A litter of 14 Sprague-Dawley rats, (from Charles River, St-Constant, Canada), obtained at 5 weeks old are accommodated individually in cages. The animals are acclimatized to a temperature of 22° C.+/−1° C. and constant humidity and airflow conditions. The animals are fed a high sucrose diet. During the 2 weeks prior to experimentation, the animals have free access to tap water, are provided their controlled diet and their body weight and food intake are recorded every other day. The high-sucrose diet consists of 62.5% (wt/wt) sucrose, 6.5% corn oil, 20% protein (casein, purified high nitrogen), 0.3% dl-methionine, 1% vitamin mix, 4.7% mineral mix and 5% cellulose. The energy density of the high-sucrose diet is 16.81 kJ/g. This high-sucrose diet induces insulin resistance. The animals are starved for 12 hours prior to testing. Seven of the animals receive up to one unit of kallikrein by a bolus IV injection and the untreated animals are given a bolus IV injection of saline solution. The euglycemic clamp protocol is performed to acquire blood samples.

Analytical Methods—The rats are anesthetized and venous blood samples are taken for plasma glucose and insulin assessment and rapidly centrifuged. The plasma is either immediately assayed or stored at −20° C. and examined within 3 days. Resting heart rate, blood pressure and regional blood flows are recorded over 30 minutes in quiet, unrestrained and unsedated rats.

Euglycemic Clamp Methodology—All rats receive an infusion of regular porcine insulin at a rate of 16 mU·kg⁻¹·min⁻¹ for 2 hours. Ten minutes after the insulin infusion started, a 200 g/L glucose solution is infused at variable rates to maintain blood glucose at the preclamp level according to frequent arterial blood glucose determinations performed at 10-minute intervals. The Euglycemic hyperinsulinemic clamp is performed over 2 hours, while blood pressure, heart rate, and regional blood flow is measured continuously with blood samples. The amount of glucose required to maintain euglycemia during the last hour of the clamp, corresponds to the steady-state concentration of insulin, used as an index of insulin sensitivity. This index is measure as the area under the curve.

Results and Discussion—The control animals maintain the same glucose tolerance and the area under the curve remains unchanged. The kallikrein treated rats have a greater area under the curve due to increased insulin sensitivity.

### Example Two

### Tissue Kallikrein and Glucagon-Like Peptide 1 (GLP-1) Combination Therapy Lowers Plasma Glucose in Synergistic Manner in Animal Model Following Glucose Challenge

Groups of 5 male Wistar rats weighing 200+/−10 g were employed. Fasted animals were injected with agent or placebo (GLP-1, 1 nmole/kg, iv) and/or gavaged with agent or placebo (tissue kallikrein (SIGMA), 200 U) one hour prior to glucose challenge. A blood sample was taken immediately prior oral glucose loading (2 g/kg). At 90 minutes post glucose challenge a second blood sample was taken. The serum glucose samples were assessed by enzymatic method (Mutaratase-GOD) for the time points.

Results and Discussion—Animals treated with GLP-1 alone were administered a suboptimal dosage of GLP-1. As expected, animals treated with GLP-1 alone responded to the glucose challenge in a similar manner as the placebo treated animals (see Table 1). Animals treated with tissue kallikrein alone were also administered a suboptimal dosage. As expected, animals treated with tissue kallikrein alone responded to the glucose challenge in a similar manner as the placebo treated animals (see Table 1). In contrast, animals treated with the same dosages of GLP-1 and tissue kallkrein in combination showed significantly lowered blood glucose levels as compared to the placebo treated animals (see Table 1). The results indicate that tissue kallikrein and GLP-1 act together in a synergistic manner to lower blood glucose levels.

TABLE 1
Glucose lowering effects of Tissue Kallikrein (KLK1) and GLP-1.

| | | | | | Change in |
|---|---|---|---|---|---|
| | | | Blood Glucose | Blood Glucose | Blood Glucose |
| Treatment | Dose | Route | at T₀ (mg/dL) | at T₉₀ (mg/dL) | (mg/dL) |
| placebo | 10 ml/kg | PO | 110 | 222 | 112.3 ± 19.3 |
| | (2% | | | | |
| | Tween 80) | | | | |
| GLP-1 | 1 nmol/g | IV | 110 | 216 | 116.1 ± 18.1 |
| | (0.9% | | | | |
| | NaCl) | | | | |
| tissue kallkrein | 200 U | PO | 109 | 229 | 120.2 ± 20.7 |
| | (PBS) | | | | |
| GLP-1 + tissue | As above | As above | 119 | 201 | 80.8 ± 20.8 |
| kallikrein | | | | | |

The differences between all the controls versus the combination (KLK1 + GLP) are significantly different at the 95% (t = 2.262) confidence interval.

1-34. (canceled)

35. A method for the treatment of insulin resistance in a subject with diabetes, comprising administering to the subject (a) a tissue kallikrein (KLK1) polypeptide and (b) an incretin or incretin mimetic.

36. The method of claim 35, where the subject has type 2 diabetes.

37. The method of claim 35, where the KLK1 polypeptide is the mature polypeptide of KLK1.

38. The method of claim 37, where the KLK1 polypeptide is the mature polypeptide of human KLK1.

39. The method of claim 38, where the mature polypeptide of human KLK1 comprises residues 25-262 of SEQ ID NO:2.

40. The method of claim 35, where the incretin or incretin mimetic is a glucagon like peptide-1 (GLP-1) or an analogue thereof.

41. The method of claim 40, where the analogue is exenatide, CJC-1131, liraglutide, ZP10, BIM51077, LY315902, or LY307161.

42. The method of claim 35, where (a) and (b) are administered as separate formulations.


SEQUENCE LISTING (2 sequences)

SEQ ID NO: 1 - protein, 263 aa, Sus scrofa
MWSLVMRLALSLAGTGAAPPIQSRIIGGRECEKDSHPWQVAIYHYSSFQCGGVLVDPKWVLTAAHCKNDNYQVWLGRHNLFENEVTAQFFGVTADFPHPGFNLSLLKNHTKADGKDYSHDLMLLRLQSPAKITDAVKVLELPTQEPELGSTCQASGWGSIEPGPDDFEFPDEIQCVELTLLQNTFCADAHPDKVTESMLCAGYLPGGKDTCMGDSGGPLICNGMWQGITSWGHTPCGSANKPSIYTKLIFYLDWINDTITENP

SEQ ID NO: 2 - protein, 262 aa, Homo sapiens
MWFLVLCLALSLGGTGAAPPIQSRIVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTEEPEVGSTCLASGWGSIEPENFSFPDDLQCVDLKILPNDECKKAHVQKVTDFMLCVGHLEGGKDTCVGDSGGPLMCDGVLQGVTSWGYVPCGTPNKPSVAVRVLSYVKWIEDTIAENS